(19) United States
(12) Patent Application Publication
(10) Pub. No.: US 2006/0018967 A1
(43) Pub. Date: Jan. 26, 2006

(54) Method for treating pipelines

(76) Inventor: Otto Schildhauer, Weinheim (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 11/203,886

(22) Filed: Aug. 15, 2005

Related U.S. Application Data
(63) Continuation of application No. PCT/DE03/04169, filed on Dec. 17, 2003.

(30) Foreign Application Priority Data
Feb. 18, 2003 (DE) 103 06 842.2

(51) Int. Cl. B05D 3/00; A61K 9/14

(52) U.S. Cl. 424/486; 427/2.3

(57) ABSTRACT

A method for treating and sanitizing a pipeline, such as a drinking water line, wherein the pipeline is freed from internal deposits, if any, and then internally covered with a coating material. The coating material may be easily applied to the interior of the pipeline by the disclosed methods, and to insure easy hygienic conditions in the interior of the pipeline on a long-term basis, the coating material is mixed with an agent that is harmful to lesser developed organisms and harmless to higher developed organisms, i.e., an agent with antimicrobial properties.

### CROSS REFERENCE TO RELATED APPLICATION

The present application is a continuation of international application PCT/DE2003/004169, filed 17 Dec. 2003, and which designates the U.S. The disclosure of the referenced application is incorporated herein by reference.

### BACKGROUND OF THE INVENTION

The present invention relates to a method for treating pipelines, in particular for sanitizing drinking water lines, wherein the pipeline, if necessary, is freed from internal deposits and then internally covered with a coating material.

Methods of this kind for sanitizing pipelines have been known from practice for a long time, for example, from DE 40 34 483, DE 44 30 334 A1, as well as DE 44 04 473 C2. These prior documents disclose generic methods for sanitizing pipelines, wherein heavy contamination and/or corrosion are removed from the inside wall of the pipeline. Corrosion has often led to pitting or holes in the pipeline, so that the required impermeability no longer exists.

Methods of the generic kind are carried out in practice in many different ways. In all commonly used methods, one removes in a first step, mechanically and/or chemically, the internal deposits from the inside wall, and covers the latter with a coating material, if need be, after further treatment steps. In a physical respect, the generic methods are fully developed. In most cases, they ensure excellent impermeability and little flow resistance to the water flowing through the lines. However, on a long-term basis, it cannot be ensured that the pipelines correspond with respect to hygiene to the required DIN standards [German Industrial Norms] and the associated hygienic conditions, since the media carried in the interior of the pipelines, normally tap water, bring impurities into the pipelines on the one hand, and since impurities form in the entered media on the other hand, which occurs in particular, when media have not moved through the pipelines for a long time.

Such impurities and unhygienic conditions are especially problematic in the interior of drinking water lines. Infectious germs may form primarily in lines that carry drinking water heated to temperatures from about 30° C. to 45° C. For example, it is known that legionellae are quite a natural constituent of all fresh waters and are largely harmless in low concentrations. However, when the water is heated to a range from 35° C. to 45° C., colonies of legionellae will be able to develop. In this process, it is possible that the water becomes contaminated to such an extent that an increased risk of infection arises when the water is used or water vapor is inhaled. An increased risk of infections exists specifically in hospitals or preschools, where persons having in part a weakened immune defense system may use the water. This situation is especially problematic in that on the inside surfaces of the pipeline, sediments or biofilms may form, in which legionellae settle with stubbornness.

While the known methods permit cleaning and sealing defective or contaminated pipelines in a reliable manner, they are unable to ensure hygienic conditions in the interior of the pipelines on a long-term basis.

It is therefore an object of the present invention to provide a method for sanitizing pipelines, in particular drinking water lines, which is easy to apply and ensures hygienic conditions in the interior of the pipeline on a long-term basis.

### SUMMARY OF THE INVENTION

The above and other objects and advantages of the invention are achieved by the provision of a method for sanitizing pipelines, in particular drinking water lines, wherein the pipeline is freed from internal deposits and covered in its interior with a coating material, and wherein the coating material is mixed with an agent that is harmful to lesser developed organisms and harmless to higher developed organisms, i.e., an agent with antimicrobial properties such as a biocide or nanosilver.

In accordance with the invention, it has been found that the addition of such an agent to the coating material produces a homogeneous distribution of the agent on the inner surface of the pipe. The homogeneous distribution of the agent ensures that microbes are destroyed, which settle on the inner surface of the pipe in the form of sediment formation or biofilms. In this respect, hygienic conditions exist in the interior of the pipe, which effectively prevent the formation of bacteria, yeasts, fungi, or algae on a long-term basis. Thus, the method of the invention makes sure that while higher developed animals with a backbone, vertebrates, and in particular humans are not harmed by the agent, lesser developed organisms are reliably destroyed.

### DETAILED DESCRIPTION OF THE PREFERRED EMBODIMENTS

In one preferred embodiment of the invention, the antimicrobial agent comprises a biocide that is added to the coating material. Biocides are substances or preparations, which are destined to have the inherent property to destroy organisms or to restrict them at least in their vital function. This property is common to biocides and plant protectants. The essential difference from plant protectants lies in that biocides are not used for agricultural applications, but for harmful organisms, which jeopardize the wellbeing of the human or its goods. It is possible and very advantageous to use biocides that have a selective effect and destroy only harmful organisms, but are harmless to humans or house animals.

As a specially suited biocide, it would be possible to use so-called SAM polymers which are highly effective against bacteria, fungi, and algae. SAM polymers are active against microbes, but totally harmless to higher developed organisms. They destroy bacteria, such as legionellae, and prevent mold fungi from settling on surfaces. The SAM polymers have a helical structure, whereby they split up their functional group outwardly. In this manner, a high electrostatic effect develops, which effectively prevents micro-organisms from settling. The name SAM stands for “Sustainable Active Microbicidal.”

As antimicrobially active agent, it would be possible to use a substance that is suited for an ion exchange with water, so as to prevent formation of a biofilm. This substance may be silver or a silver-containing material. Of special advantage is the use of nanosilver, which permits imparting to the coating material an antimicrobial effect. Such an antibacterial coating is to be preferred at any rate over directly adding a dosage of silver ions to the tap water, inasmuch as a permissible dosage in the drinking water has only a little effect in preventing a biofilm.

As coating material, it would be possible to use a bicomponent epoxy resin. The use of a bicomponent epoxy resin is advantageous in that the components, when separately stored, have an almost unlimited shelf life, and that they are mixed together only shortly before their application.

The antimicrobially active agent could be added to the binder component of the epoxy resin. The addition could occur in the form of particulate inclusions, so that in the cured state an interstitial structure is obtained with at least a slight porosity. The addition of the antimicrobial agent could also occur in the form of fibers, with the fibers serving as carrier of the antimicrobial agent or consisting as a whole of an antimicrobial agent.

All in all, as a function of the chemical structure of the biocide, it is possible and advantageous to mix the latter with the curing component or with the binder component of the epoxy resin. With that, it is ensured that the biocide does not undergo undesired combinations or reactions either with the curing component or with the binder component of the epoxy resin.

The epoxy resin could also be inserted into the pipeline as a bicomponent cartridge. The use of a bicomponent cartridge permits the user a particularly defined and purposeful insertion also into pipelines having a small diameter or a complicated geometry. With that, a particularly exact and defined working method is made possible.

The epoxy resin could be inserted at one end of the pipeline until it emerges from the other end. This working method is advantageous in that it ensures the user that the entire pipe is filled with the coating material. In this respect it is avoided that larger surfaces are not coated with the epoxy resin, and that sections of the inner surface of the pipe are thereby left uncoated.

It would be also possible to move the epoxy resin through the pipeline by suction. The movement of the epoxy resin through the pipeline by suction is advantageous in that brittle points of the pipeline are not outwardly biased by force. It is thereby avoided that a porous pipe with holes is still further damaged, in that the holes tear out and widen.

When the pipe is constructed adequately stable, it will be possible to blow or press the epoxy resin through the pipeline. Blowing or pressing the epoxy resin through the pipeline is advantageous in that it is also possible to force highly viscous materials through the pipeline under a high pressure.

A particularly variable coating method could be carried out by alternately sucking and blowing or pressing the epoxy resin through the pipeline. This procedure would permit wetting stable sections of the pipe with the coating material by pressing. Less stable sections could be coated by suction. With that, it is possible to adapt in different sections the load of the pipe to the stability of the pipe.

In a technically problem free and commercially easily realizable manner, one could reciprocate the epoxy resin by alternating overpressure and vacuum with the use of pumps. The method is additionally advantageous in that hard to wet places could be coated by being repeatedly brought into contact with the coating material. Naturally, it is also possible to reciprocate the epoxy resin by alternating the overpressure at both ends of the pipeline. All in all, as a function of the stability of the pipeline, the user can flexibly and variably select between overpressure and vacuum for purposes of moving the epoxy resin in the pipeline.

Naturally, it is possible to perform the foregoing method steps with any coating material, whose fluid properties permit carrying out the described steps. In this respect, the method steps are not limited to the use of epoxy resin.

To remove stubborn internal deposits, it would be possible to use abrasives. The use of abrasives represents an effective method of removing in a reliable manner from the pipeline both corrosive residues and impurities caked to the pipeline, since the abrasives abrade by their roughness the inner surface of the pipe.

As regards a very environmentally protective realization of the method, it will be advantageous to use as abrasive sand or corundum, since sand or corundum are ecologically harmless. The abrasive used to remove internal deposits could be particles of any kind, which would however have to be harder than the internal deposits being removed. In this connection, it has been found satisfactory to use for sanitizing pipeline particles of a grain size in a range from 0.3 mm to 6 mm. A specific weight of more than 3.0 grams per cubic centimeter has likewise turned out to be advantageous. In this case, air is used as carrier medium. Concretely, the abrasively active particles could be corundum or quartz sand. Likewise, it would be possible to make the abrasively active particles of metal particles, in particular ferromagnetic particles. The use of this type of particles would permit a subsequent magnetic separation of the abrasively active particles from the separated internal deposits which consist of rust and/or lime.

The abrasive particles could be entrained in a flow medium. This would permit bringing all zones of the pipeline, in particular also angled sections, into contact with the abrasive in a reliable manner, since the abrasives are transported without difficulties by the flow medium.

A particularly protective treatment of the pipeline could occur by moving the flow medium through the pipeline by suction. The suction prevents the pipe walls from being outwardly biased by force, so that porous and thin places of the pipe wall are protected against tearing up and widening.

In the case of very stubborn contaminations and stable pipelines, it would also be possible to blow or press the flow medium through the pipeline. Blowing and pressing have the advantage that they permit applying high pressures, which cause the abrasive to be pressed at a high speed not only along the pipeline, but also in the radial direction against the inside wall of the pipe.

All in all, as a function of the pipe quality, the user could move the flow medium through the pipeline by alternating suction and blowing. This procedure gives the user the possibility of applying in different regions of the pipeline differently powerful cleaning methods.

The movement of the abrasives inside the pipelines could be generated in an advantageous manner in that the flow medium and with that the abrasives are caused to reciprocate in the pipeline by alternating vacuum and overpressure. In other words, one could attain in the pipeline alternately a vacuum and an overpressure that cause the abrasive to be alternately sucked and blown through the pipe. However, it is likewise possible to reciprocate the flow medium in the pipeline by alternating the vacuum at both ends of the pipeline. In this instance, one would have the above described advantages. Finally, it is also possible to reciprocate the flow medium in the pipeline by alternating the overpressure at both ends of the pipeline. In this case, the overpressure can be purposefully used for transporting the abrasive only when there are no significant leakages in the pipeline.

For the purposes of an environmental compatibility of the method, one could use as flow medium water, which is both easily available and easy to remove.

One could also use air as the flow medium. This will be advantageous to the extent that there are no removal problems with respect to the flow medium, since the latter disappears after its use.

As regards a highly environmentally protective working of the method, it would be possible to filter out the abrasives after removing the internal deposits from the flow medium. To this end, one could use a filtering device. In this case, it will be possible to remove the flow medium together with the abrasive from the pipeline by suction with the use of a compressor that is followed by a filtering device.

A particularly effective cleaning could be attained with the use of ultrasound. In so doing, the internal deposits formed on the inside wall of the pipeline are advantageously separated from the inside wall of the pipeline by means of ultrasound, and they will be crushed, if need be. For removing the internal deposits, this procedure does not require a priori the use of any abrasives, since the ultrasound impacting upon the internal deposits separates same from the inside wall of the pipeline and, moreover, causes same to be crushed. Subsequently, these internal deposits can be effortlessly removed from the pipeline. As regards a concrete working of this ultrasonic cleaning, one could insert into the pipeline an ultrasonic probe. For an effective ultrasonic treatment of the entire pipeline, the ultrasonic probe could be guided or moved through the pipeline substantially over the entire length thereof. This movement could occur, for example, by means of a flexible shaft. However, it would be likewise possible to suck the ultrasonic probe directly into the pipeline, i.e., right to the outlet end of the pipeline. The ultrasonic probe itself could include a miniaturized ultrasonic exciter, which emits ultrasound approximately orthogonally to the direction of movement, i.e., transversely to the longitudinal extension of the pipeline. This would ensure that the ultrasonic vibrations directly impact upon the internal deposits, with a damping of the ultrasonic vibrations being largely prevented because of the short distances.

After the removal of the internal deposits, the pipeline could be heated. The application of the coating material, and thus on the one hand an excellent wetting with the inside wall of the pipeline and on the other hand a rapid subsequent curing, is especially favored by heating the pipeline after removing the internal deposits to a temperature above room temperature. In this connection, a temperature of about 40° C. has been found especially satisfactory. The heating of the pipeline could again occur by sucking heated air through the pipe until the desired temperature is reached at the outlet end. This temperature, in turn, could be detected via the temperature drop between the inlet end and the outlet end of the pipeline. Essential, however, is that the heating of the pipeline occur by sucking therethrough heated air and not by forcing therethrough compressed air. Finally, the preheated air used for heating the pipeline could likewise be removed by suction via a filtering device. This filtering device can directly precede a compressor that is used for taking in the air.

Many modifications and other embodiments of the inventions set forth herein will come to mind to one skilled in the art to which these inventions pertain having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method for sanitizing a pipeline, such as a drinking water line, comprising the steps of
freeing internal deposits, if any, from the interior of the pipeline, and
covering the interior of the pipeline with a coating material which is mixed with an antimicrobial agent that is harmful to lesser developed organisms and harmless to higher developed organisms, and wherein the antimicrobial agent comprises a biocide.

2. The method of claim 1, wherein the biocide comprises an antimicrobially active SAM polymer.

3. The method of claim 1, wherein the antimicrobial agent is added to a binder component of an epoxy resin.

4. The method of claim 3, wherein the coating material is in the form of particulate inclusions.

5. The method of claim 3, wherein the coating material is in the form of fibers, which serve as a carrier of the antimicrobial agent.

6. The method of claim 1, wherein the coating material comprises an epoxy resin which is inserted into the pipeline by means of a cartridge.

7. The method of claim 1, wherein the pipeline has opposite ends, and wherein the coating material is inserted into one end, until it emerges from the other end.

8. The method of claim 1, wherein the coating material is alternately sucked and/or blown through the pipeline.

9. The method of claim 8, wherein the coating material is reciprocated in the pipeline by alternating overpressure and vacuum, or by alternating either an overpressure or a vacuum at the ends of the pipeline.

10. The method of claim 1 wherein the freeing step includes the use of an abrasive for removing internal deposits.

11. The method of claim 10, wherein the abrasive comprises sand or corundum and is entrained in a flow medium which is passed through the pipeline.

12. The method of claim 11, wherein the flow medium is reciprocated in the pipeline by alternating overpressure and vacuum, or by alternating either an overpressure or a vacuum at the ends of the pipeline.

13. The method of claim 11, wherein the flow medium comprises water or air.

14. The method of claim 11, wherein the abrasive is filtered out from the flow medium after removing the internal deposits.

15. The method of claim 1, wherein the freeing step includes the use of ultrasound.

16. The method of claim 1, comprising the further step of heating the pipeline after removing the internal deposits.

17. A method for sanitizing a pipeline, such as a drinking water line, comprising the steps of
freeing internal deposits, if any, from the interior of the pipeline, and
covering the interior of the pipeline with a coating material which is mixed with an antimicrobial agent that is harmful to lesser developed organisms and harmless to higher developed organisms, and wherein the antimicrobial agent comprises nanosilver in a form suited for ion exchange with water.

18. The method of claim 17, wherein the antimicrobial agent is added to a binder component of an epoxy resin.

19. The method of claim 18, wherein the coating material is in the form of particulate inclusions.

20. The method of claim 18, wherein the coating material is in the form of fibers, which serve as a carrier of the antimicrobial agent.

21. The method of claim 17, wherein the coating material comprises an epoxy resin which is inserted into the pipeline by means of a cartridge.

22. The method of claim 17, wherein the pipeline has opposite ends, and wherein the coating material is inserted into one end, until it emerges from the other end.

23. The method of claim 17, wherein the coating material is alternately sucked and/or blown through the pipeline.

24. The method of claim 23, wherein the coating material is reciprocated in the pipeline by alternating overpressure and vacuum, or by alternating either an overpressure or a vacuum at the ends of the pipeline.

25. The method of claim 17 wherein the freeing step includes the use of an abrasive for removing internal deposits.

26. The method of claim 25, wherein the abrasive comprises sand or corundum and is entrained in a flow medium which is passed through the pipeline.

27. The method of claim 26, wherein the flow medium is reciprocated in the pipeline by alternating overpressure and vacuum, or by alternating either an overpressure or a vacuum at the ends of the pipeline.

28. The method of claim 26, wherein the flow medium comprises water or air.

29. The method of claim 26, wherein the abrasive is filtered out from the flow medium after removing the internal deposits.

30. The method of claim 17, wherein the freeing step includes the use of ultrasound.

31. The method of claim 17, comprising the further step of heating the pipeline after removing the internal deposits.
